(19) United States
(12) Patent Application Publication
(10) Pub. No.: US 2026/0306054 A1
(43) Pub. Date: Oct. 8, 2026

(54) SYSTEMS AND METHODS FOR EVALUATING PULMONARY DISEASE USING ACOUSTIC AIRWAY MEASUREMENTS FROM A SMARTPHONE

(71) Applicant: UNIVERSITY OF PITTSBURGH - OF THE COMMONWEALTH SYSTEM OF HIGHER EDUCATION, PITTSBURGH, PA (US)

(72) Inventors: WEI GAO, MARS, PA (US); WEI CHEN, SEWICKLEY, PA (US); ERICK FORNO, INDIANAPOLIS, IN (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 19/473,939

(22) PCT Filed: Jun. 14, 2024
(86) PCT No.: PCT/US2024/034071

§ 371 Date: Oct. 9, 2025

Related U.S. Application Data
(60) Provisional application No. 63/508,655, filed on Jun. 16, 2023.

(51) Int. Cl. A61B 8/12; A61B 8/00; A61B 8/08

(52) U.S. Cl. CPC A61B 8/12; A61B 8/08; A61B 8/42; A61B 8/4427; A61B 8/5223

(57) ABSTRACT

A method of determining the presence and/or severity of a pulmonary disease in an individual first includes directing an ultrasound signal generated by a speaker of an device into an airway of the individual through an interface coupled to the device. The interface includes a mouthpiece to be received in a mouth of the individual. The method further includes receiving with a microphone of the device a reflected version of the ultrasound signal from the airway and determining from the reflected version of the ultrasound signal the presence and/or severity of the pulmonary disease in the individual. The presence and/or severity of the pulmonary disease is determined using a trained machine learning model of a controller of the device

### CROSS REFERENCE TO RELATED APPLICATIONS

This application claims priority to U.S. Provisional Application No. 63/508,655 filed Jun. 16, 2023, titled “SYSTEMS AND METHODS FOR EVALUATING PULMONARY DISEASE USING ACOUSTIC AIRWAY MEASUREMENTS FROM A SMARTPHONE”, the contents of which are incorporated in its entirety herein by reference.

### STATEMENT OF GOVERNMENT SUPPORT

This invention was made with government support under grant #2205360 awarded by the National Science Foundation (NSF). The government has certain rights in the invention.

### FIELD OF THE INVENTION

The present invention relates to systems and methods for pulmonary function testing, and, in particular, to systems and methods for estimating lung function, calculating airway mechanics and/or detecting airway obstructions using a smartphone or similar device, and from such calculating/detecting determining the presence and/or severity of pulmonary disease in a subject. The present invention further relates to interfaces for use in such systems and methods.

### BACKGROUND OF THE INVENTION

Pulmonary diseases, such as asthma and chronic obstructive pulmonary disease (COPD), were the fourth cause of death in the US before the COVID-19 pandemic and hence are a major public health issue. Diagnosis and management of these diseases are often based on subjective symptom reports by patients. However, patients typically fail to recognize early small symptoms or slow decline in lung function with chronic diseases, especially when being out of clinic. This poor perception leads to acute exacerbations resulting in emergency department visits and hospitalizations. Evaluating pulmonary diseases remotely but objectively via telemedicine, hence, is crucial to disease management, both acutely and in the long term.

Telemedicine has enormous potential to improve pulmonary disease evaluation and symptom control. These advantages are particularly important in situations such as the COVID-19 pandemic, with pulmonary patients unable or unwilling to attend clinic visits or use shared equipment. However, current telemedicine has mostly been limited to video calls that still rely on subjective symptom self-report, with limited or no capability of objectively examining airway conditions.

In attempting to address such deficiencies, current sensing techniques either attach force sensors, ultrasound sensors or inductive bands onto the human body, or use expensive arrangements such as infrared cameras, depth cameras or RF systems. However, the requirement of such arrangements for extra hardware results in limited usability in long-term telemedicine. Using smartphones for sensing can address this limitation, but most existing solutions are limited to monitoring breath rates or respiratory events (e.g., apnea), which are not directly related to pulmonary disease evaluation. Other techniques measure lung function externally by passively overhearing the breathing sounds or actively measuring chest mobility, but such techniques cannot examine the airway's alternations of internal physiological conditions, such as airway obstruction and restriction caused by inflammation and mucus hypersecretion, which are crucial to pulmonary disease evaluation.

### SUMMARY OF THE INVENTION

As one aspect of the present invention a method of determining the presence and/or severity of a pulmonary disease in an individual is provided. The method comprises: directing an ultrasound signal generated by a speaker of an electronic device into an airway of the individual through an interface coupled to the device, wherein the interface has a mouthpiece structured to be received in a mouth of the individual; receiving with a microphone of the electronic device a reflected version of the ultrasound signal from the airway; and determining from the reflected version of the ultrasound signal the presence and/or severity of the pulmonary disease in the individual, wherein the presence and/or severity of the pulmonary disease is determined using a trained machine learning model of a controller of the electronic device.

Directing the ultrasound signal generated by a speaker of an electronic device into an airway of the individual may comprise directing a series of acoustic pulses into the airway, and wherein receiving with the microphone of the device the reflected version of the ultrasound signal from the airway comprises receiving a reflected version of the series of acoustic pulses from the airway.

The method may further comprise filtering and denoising the reflected version of the ultrasound signal with the controller to produce an adjusted reflected signal, and the presence and/or severity of the pulmonary disease in the individual may then be determined by the controller from the adjusted reflected signal.

The electronic device may comprise a smartphone.

The mouthpiece may comprise: a body; a passage defined within, and extending through the body from an inlet that is coupled to the second end of the interface tube to an outlet opposite the inlet; and an incisor stopper disposed adjacent the inlet, the incisor stopper being structured to engage incisors of the individual to fix the orientation of the body and thus the mouthpiece when the individual bites down on the body. The mouthpiece may further comprise a tongue depressor positioned at the bottom of the body adjacent the outlet that is positioned and structured to be engaged by a tongue of the individual. The body, the incisor stopper, and the tongue depressor may each be portions of a unitary member.

As another aspect of the present invention, a system for determining the presence and/or severity of a pulmonary disease in an individual is provided. The system comprises: an electronic device comprising: a housing; a speaker positioned on or in the housing; a microphone positioned on or in the housing; and a controller in communication with the speaker and the microphone; and an interface structured to convey ultrasound signals produced by the speaker to the airway of the individual and from the airway of the individual to the microphone, wherein the controller is structured and configured for: transmitting an ultrasound signal from the speaker to an airway of the individual via the interface; receiving from the microphone a reflected version of the ultrasound signal from the interface reflected from the airway of the individual; and determining from the reflected signal the presence and/or severity of the pulmonary disease in the individual, and wherein the controller is structured and configured for determining the presence and/or severity of the pulmonary disease from the reflected signal using a trained machine learning model.

The controller may be further structured and configured for filtering and denoising the reflected version of the ultrasound signal to produce an adjusted reflected signal and for determining the presence and/or severity of the pulmonary disease in the individual from the adjusted reflected signal.

The electronic device may be a smartphone.

The interface may comprise: an adaptor having a body generally defining an interior void therein, the body having a first portion and a second portion, the first portion being engaged on and around an end of the electronic device such that openings in the housing for the speaker and the microphone are encompassed by body and open into the interior void; a mouthpiece structured to be positioned in the mouth of the individual; and an interface tube having a first end coupled to the second portion of the adaptor and an opposite second end coupled to the mouthpiece. The mouthpiece may comprise: a body; a passage defined within, and extending through the body from an inlet that is coupled to the second end of the interface tube to an outlet opposite the inlet; and an incisor stopper disposed adjacent the inlet, the incisor stopper being structured to engage incisors of the individual to fix the orientation of the body and thus the mouthpiece when the individual bites down on the body. The mouthpiece may further comprise a tongue depressor positioned at the bottom of the body adjacent the outlet that is positioned and structured to be engaged by a tongue of the individual. The body may comprise a unitary member and the body, the incisor stopper, and the tongue depressor may each be portions of the unitary member.

As yet another aspect of the present invention, an interface for use with an electronic device for determining the presence and/or severity of a pulmonary disease in an individual is provided. The interface comprises: a mouthpiece structured to be positioned in the mouth of the individual, the mouthpiece comprising: a body; a passage defined within, and extending through the body from an inlet to an outlet opposite the inlet; and an incisor stopper disposed adjacent the inlet, the incisor stopper being structured to fix the orientation of the body and thus the mouthpiece when the individual bites down on the body.

The mouthpiece may further comprise a tongue depressor positioned at the bottom of the body adjacent the outlet and that is structured to be engaged by the tongue of the individual. The body may comprise a unitary member and the body, the incisor stopper, and the tongue depressor may each be portions of the unitary member.

The interface may further comprise: an adaptor having a body generally defining an interior void therein, the body having a first portion and a second portion, the first portion structured to be engaged on and around an end of the electronic device such that openings in the housing for the speaker and the microphone are encompassed by the body and open into the interior void; and an interface tube having a first end coupled to the second portion of the adaptor and an opposite second end coupled to the inlet of the mouthpiece.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of an electronic device in accordance with one example embodiment of the present invention which may be employed in systems out methods in accordance with example embodiments of the present invention;

FIG. 2 is a system for determining the presence and/or severity of a pulmonary disease in an individual in accordance with an example embodiment of the present invention;

FIGS. 3 and 4 are perspective views of an adaptor in accordance with one example embodiment of the present invention for use in a system such as shown in FIG. 2;

FIGS. 5 and 6 are perspective views of a mouthpiece in accordance with one example embodiment of the present invention for use in a system such as shown in FIG. 2;

FIG. 7 is a side elevation view of the mouthpiece of FIGS. 5 and 6;

FIG. 8 is a top view of the mouthpiece of FIGS. 5-7;

FIG. 9 is a bottom view of the mouthpiece of FIGS. 5-8;

FIG. 10 is an end view of the mouthpiece of FIGS. 5-9 looking at an end of the mouthpiece that opens into the mouth of an individual when the mouthpiece is positioned in the mouth of the individual;

FIG. 11 is a partially schematic view of the mouthpiece of FIGS. 5-10 shown positioned in a sectional anatomical view of a mouth of an individual in accordance with one example embodiment of the present invention;

FIG. 12 is an analytical modeling of a source of reflection in accordance with an example embodiment of the present invention;

FIG. 13 is a reference airway CSA curve in accordance with an example embodiment of the present invention;

FIG. 14 is a general overview of machine learning for pulmonary disease evaluation in accordance with an example embodiment of the present invention; and

FIGS. 15A-15D are a series of displays provided on a touchscreen of an electronic device in accordance with one example embodiment of the present invention.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the term “connected” shall mean that elements are electrically connected such that signals may pass from one of the elements to the other.

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Embodiments of the present invention will now be described, for purposes of explanation, in connection with numerous specific details in order to provide a thorough understanding of the subject invention. It will be evident, however, that the present invention can be practiced without these specific details without departing from the spirit and scope of the present invention.

Alterations of the internal physiological conditions of a subject's airway are a fundamental part of many pulmonary diseases, and can be reflected by corresponding changes in airway caliber. In asthma for example, airway inflammation causes swelling and acute bronchoconstriction, leading to narrowing that causes symptoms and exacerbations. Severe asthma can lead to airway remodeling and more permanent narrowing. COPD is partly caused by progressive inflammatory damage to airways and alveoli (the tiny air sacs in the lungs that perform gas exchange), leading to airway obstruction and decreased lung recoil, both affecting lung function. In cystic fibrosis (CF), abnormally thick and sticky mucus clogs the airways and allows bacteria to grow, leading to chronic inflammation and recurrent infections. Embodiments of the present invention measure changes in airway caliber as the indicator of pulmonary disease conditions.

Current pulmonary disease evaluations are mostly based on pulmonary function tests (PFTs). Spirometry, as the most commonly used PFT, uses forced breathing efforts to measure breath volumes and velocities under maximum exhalation, and produce lung function indices including: 1) forced vital capacity (FVC), 2) forced expiratory volume in 1 second (FEV1), and 3) the ratio of FEV1 to FVC (FEV1/FVC). Since lung function varies greatly among individuals, clinicians categorize subjects into subgroups according to their demographics (e.g., age, gender, race, etc.), and convert the raw values of spirometry indices into percentiles in each subgroup. Typically, significantly low percentiles (<70%) are the key indicators of pulmonary diseases. However, forced maneuvers in spirometry make it difficult to be used in telemedicine without professional coaching, and spirometers in home use are known to be highly inaccurate. Embodiments of the present invention aim to provide effortless airway examination methods that do not require any forced maneuvers or difficult protocols.

Some techniques have been developed to replace the forced maneuvers in spirometry by actively transmitting acoustic waves to probe the internal conditions of the airway. For example, forced oscillation technique (FOT) and impulse oscillometry (IOS) use pressure waves to measure the airway's overall resistance and impedance, but cannot provide detailed information about the conditions of different airway segments.

The acoustic reflection technique (ART) addresses such limitation by measuring the cross-sectional areas (CSAs) at different airway positions. In such approach, the transmitted acoustic signal pulses are assumed to propagate in the airway as 1-D plane waves, which will only be reflected on the boundary between airway segments with different CSAs. Then, the CSA of the k-th airway segment (Ak) is iteratively calculated using the Ware-Aki (WA) algorithm as:

$$\frac{{A}_{k + 1}}{{A}_{k}} = \frac{( 1 - {r}_{k} )}{( 1 + {r}_{k} )}$$

where rₖ indicates the ratio between reflected and incident signals at the boundary. In practice, the WA algorithm first calculates the airway's impulse response (IR) from deconvolution between the transmitted and received signals. Then, given the Z-transform of impulse response (h(t)) as:

$$H ( z ) = {\sum }_{k = 1}^{∞} {H}_{k} {z}^{- k}$$

rₖ can be calculated from H₁, H₂, . . . , Hₖ. However, it is challenging to replicate the ART system design to commodity smartphones. An ART system uses a connecting tube to direct the acoustic signal into the airway, but the reflected signal from the airway could be reflected again by the sound source and create infinite echoes in the tube, referred to as source reflection. These echoes overlap with the airway's reflected signal and create extra measurement errors. A traditional ART system addresses this issue by placing the microphone on the tube wall to be far away from the speaker (>50 cm), to separate the airway's reflected signal and echoes in time. This solution, however, is infeasible on smartphones and similar devices where the placements of bottom speaker and microphone are very close and fixed. Hence, embodiments of the present invention utilize novel measurement protocols and signal processing algorithms, described further below in order to obtain accurate CSA measurements on smartphones and similar devices.

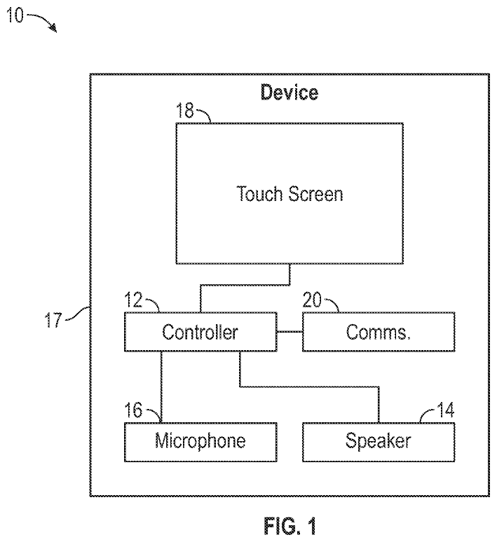

Device—An example embodiment of an electronic device 10 in accordance with embodiments of the present invention is shown schematically in FIG. 1. Device 10 includes a controller 12, a speaker 14, and a microphone 16 coupled on or in a body or housing 17 of device 10. Controller 12 is connected to, and in communication with, both of speaker 14 and microphone 16. Controller 12 is structured and configured to cause speaker 14 to emit soundwaves of predetermined frequencies and to receive information regarding soundwaves, such as those produced by speaker 14, via microphone 16. Additionally, device 10 may include one or more input and/or output devices connected to, and in communication with, controller 12, for example, without limitation, a touchscreen 18 such as shown in FIG. 1, and/or one or more communication arrangement(s) 20 in communication with controller 12 for providing input to, or communicating output from, controller 12. Such communication arrangement(s) may include, for example, without limitation, arrangements providing for wireless (e.g., Bluetooth, cellular, etc.), and/or wired communications to/from device 10 and controller 12 thereof. In the example embodiments illustrated herein, electronic device 10 is in the form of a commodity smartphone device (e.g., without limitation, an Android or iPhone device) such as readily available to the general masses. However, it is to be appreciated that device 10 may be any other suitable electronic device or devices without varying from the scope of the present invention.

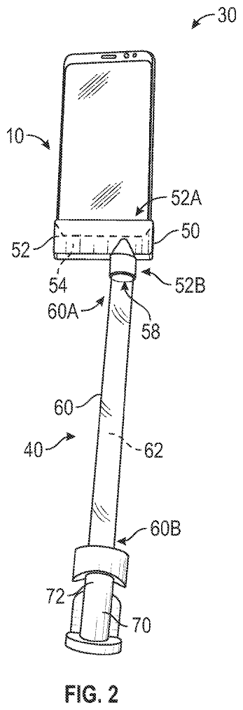

System—Embodiments of the present invention utilize such an electronic device 10 in systems for determining the presence and/or severity of a pulmonary disease in an individual (also referred to herein as a “user”, “patient”, or “subject”). FIG. 2 shows an example embodiment of such a system 30 for determining the presence and/or severity of a pulmonary disease in an individual in accordance with an example embodiment of the present invention. System 30 includes device 10 and an interface 40 for interfacing device 10 with a subject (not shown). In the example shown in FIG. 2, device 10 is shown as a smartphone device, and is employed to provide contact-less monitoring of patient lung-function, which can serve for telemedicine and to aid in the diagnosis and management of lung disease. As previously noted, it is to be appreciated that device 10 may be any other suitable device without varying from the scope of the present invention. Interface 40 includes an adaptor 50, an interface conduit 60, and a mouthpiece 70.

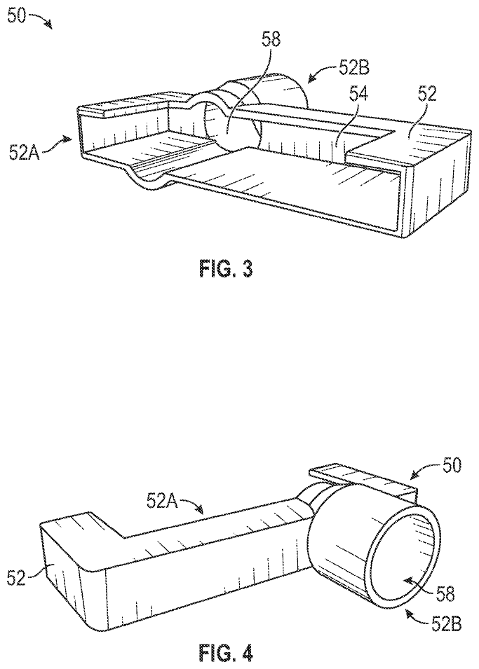

Continuing to refer FIG. 2, and additionally to FIGS. 3 and 4, adaptor 50 includes a body 52 generally defining an interior void 54 therein. Body 52 includes a first portion 52A and a second portion 52B. First portion 52A is sized and configured to engage on and around an end (not numbered) of device 10 such that openings (not numbered) in housing 17 for speaker 14 and microphone 16 are encompassed by body 52 and open into interior void 54. Second portion 52B is sized and configured to be coupled to a first end 60A of interface conduit 60. Second portion 52B of adaptor 50 includes an opening 58 which provides for fluid communication between the interior void 54 of adaptor 50 and a central passage 62 of interface conduit 60. Adaptor 52 may be formed (e.g., via 3D printing or any other suitable method(s) of manufacture) from plastic or other suitable material and may be provided with any suitable mechanism or arrangement for selectively coupling with a portion of device 10 so as to generally remain coupled with device 10 until pulled apart from device 10 with minor force (e.g., similar to the force needed to remove an average smartphone case) by a user of system 30 or other individual. In the example shown in FIG. 2, interface conduit 60 is formed from a smooth flexible tube and includes a second end 60B, opposite first end 60A, that is coupled to mouthpiece 70 via any suitable arrangement.

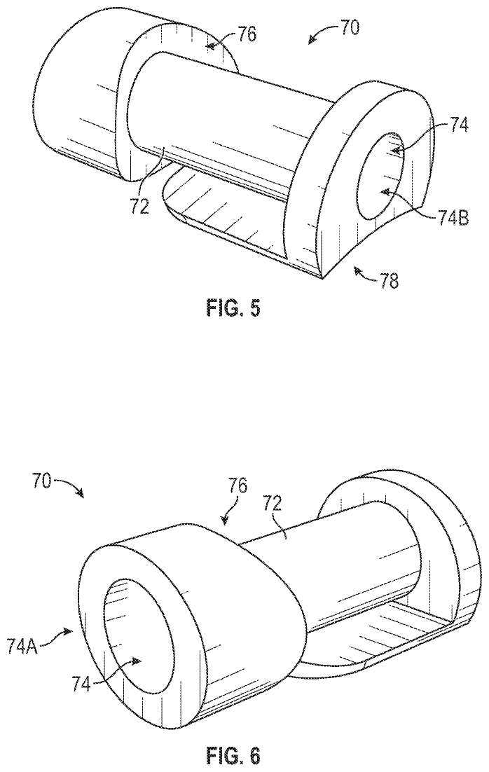

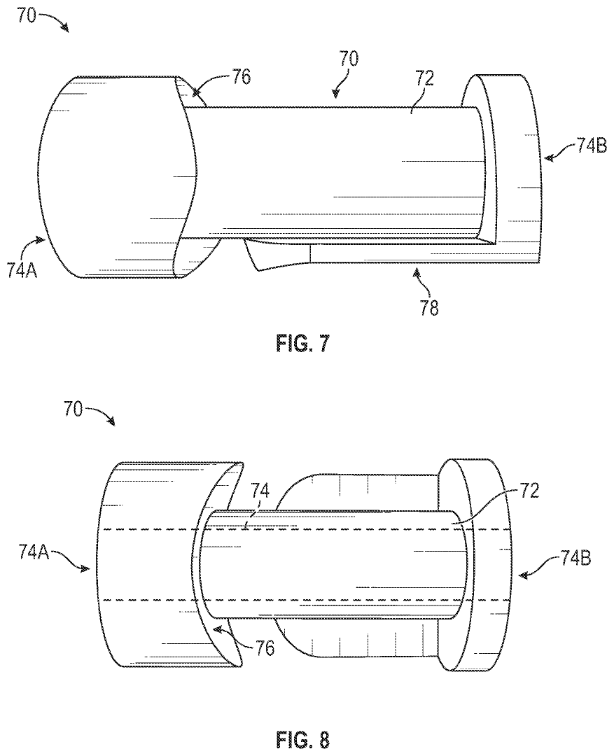

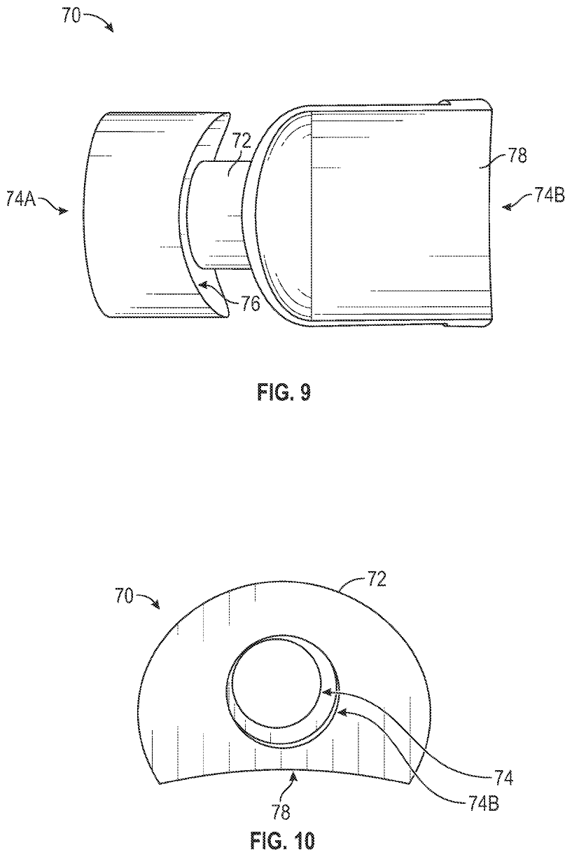

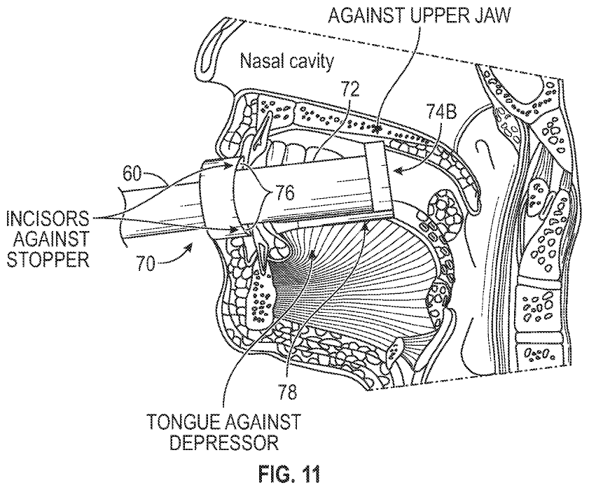

Continuing to refer to FIG. 2, as well as FIGS. 5-11, mouthpiece 70 is structured to be positioned almost entirely within a subject's mouth and includes a body 72, a passage 74 defined within, and extending through body 72 from an inlet 74A (that is structured to be coupled to second end 60B of interface conduit 60) to an outlet 74B (that opens into the mouth of the individual when the mouthpiece 70 is positioned within the mouth of an individual—e.g., see FIG. 11) opposite inlet 74A. Mouthpiece 70 further includes an incisor stopper 76 disposed adjacent the inlet 74A and a tongue depressor 78 positioned at the bottom of body 72 adjacent outlet 74B. In such example, the incisor stopper 76 is a generally vertical wall (e.g., see FIG. 7) that is concavely shaped when viewed from outlet 74B. As shown in the example of FIG. 11, the incisor stopper 76 is structured to be disposed against the incisors of an individual and fix the orientation of the body 72, and thus the mouthpiece 70, when an individual bites down on the body 72. Meanwhile, in such example the tongue depressor 78 is concavely shaped (e.g., see FIG. 10) and is structured to engage the top surface of the tongue of an individual and ensures that the outlet 74B is always oriented toward the throat of the individual when the individual is instructed to press their tongue up against the depressor, such as shown in FIG. 11. In this way, both ends of the mouthpiece 70 are fixed in the oral cavity, hence minimizing possible mobility of mouthpiece 70 during airway measurements. In the example embodiments illustrated in FIGS. 5-10, mouthpiece 70 is formed from a single unitary member (e.g., without limitation, formed from 3D printing) with each of the body 72, incisor stopper 76, and tongue depressor 78 being portions of the unitary member. It is to be appreciated that the example interface 40 may be generally created/assembled (generally at a minimal cost), coupled to a smartphone, used to interact with a patient as discussed below, uncoupled from the smartphone, and disposed of after use. It is also to be appreciated that such arrangement lends itself to very remote use as a smartphone and access to a 3D printer is all that is generally needed on-site.

Use of System—Embodiments of the present invention utilize systems, such as system 30 of FIG. 2, to determine the presence and/or severity of a pulmonary disease in an individual and provide such information to a medical practitioner for use in determining a treatment plan for the patient. To use system 30, a user first assembles system 30 by connecting the interface 40 (forming and/or assembling also if not already done) to an electronic device 10, such as a smartphone, places the mouthpiece 70 in their mouth, handholds the device 10, and breaths normally through the interface 40 via the mouthpiece 70 for a few seconds as directed by instructions provided on device 10. No forced maneuvers (e.g., deep breath and forceful exhalation), difficult protocols, or extra computing hardware are needed (beyond that of the smartphone). A software application (e.g., a smartphone “App”) executed on the device 10 provides the instructions to the user (e.g., via touchscreen 18) and causes device 10 to transmit a series of acoustic pulses (via speaker 14), which in an example embodiment each lasts 2 ms, into the airway of the user. Hundreds of airway measurements are obtained within each second, eliminating the impact of random system noise.

With the received acoustic signal, device 10 uses the WA algorithm previously described to calculate the airway's impulse response and converts it to airway CSA measurement. A prerequisite is that the acoustic signal propagation in the airway is a 1-D plane wave, and this assumption holds if the signal wavelength is smaller than twice the airway diameter. Since the diameters of most human airway structures, including trachea, pharynx, and larynx, are smaller than 3 cm, the maximum frequency of the transmitted signal is 5.7 kHz, which has a satisfactory gain on most smartphone models. Although this frequency falls in the audible bands, signal propagation is confined within the passage with >35 dB attenuation. Hence, using such approach has negligible impact on a user's comfort or health.

In order to achieve objective and precise disease evaluation, embodiments of the present invention address the impacts of possible system and human factors that may affect the accuracy of airway measurement. Afterward, these measurements are used as input to a multi-task machine learning (ML) model that evaluates pulmonary disease conditions, including the probability of disease and lung function indices.

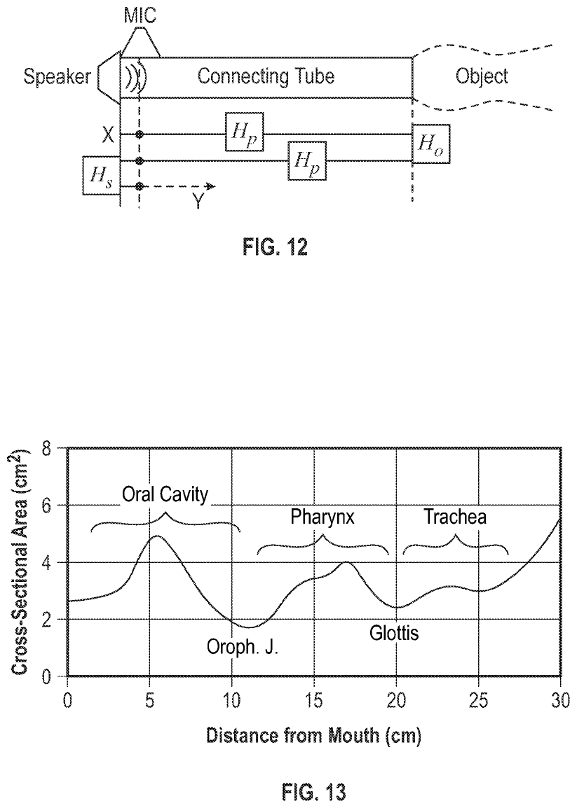

Addressing System Factors—To address the impact of source reflection previously described, we start with the analytical model of acoustic signal reflection and propagation in the interface conduit 60. When the reflected signal is a linear transformation of the incident signal without frequency shift, both the airway's reflection and source reflection are considered as linear time-invariant (LTI) systems with different transfer functions. For an input signal x(t) and the corresponding LTI system output y(t), in the complex frequency domain of Laplace transform, we have Y(s)=H(s)X(s) where H(s) is the system's transfer function. As shown in FIG. 12, we denote the transfer function of source reflection, airway's reflection, and the signal propagation in the tube as Hₛ(s), Hₒ(s) and Hₚ(s), respectively. The smartphone's received signal (Y(s)) can be written as a function of the transmitted signal (X(s)):

$$Y ( s ) = X ( s ) + {H}_{p}^{2} ( s ) {H}_{o} ( s ) X ( s ) + {H}_{p}^{2} ( s ) {H}_{o} ( s ) {H}_{s} X ( s ) + {H}_{P}^{4} ( s ) {H}_{o}^{2} ( s ) {H}_{s} ( s ) X ( s ) + … ,$$

where the high-order terms indicate the infinite echoes caused by source reflection. This can be further generalized as the following infinite geometric sequence:

$$Y ( s ) = [ X ( s ) + {H}_{p}^{2} ( s ) {H}_{o} ( s ) X ( s ) ] \cdot {\sum }_{n = 0}^{∞} {[ {H}_{p}^{2} ( s ) {H}_{o} ( s ) {H}_{s} ( s ) ]}^{n} = X ( s ) \frac{1 + {H}_{p}^{2} ( s ) {H}_{o} ( s )}{1 - {H}_{p}^{2} ( s ) {H}_{o} ( s ) {H}_{s} ( s )}$$

To calculate the impulse response hₒ(t)=L⁻¹ {Hₒ(s)} of the airway, we need to estimate X(s), Hₚ(s) and Hₛ(s), all of which only relate to the measurement system (smartphone and passage) rather than the airway. Hence, our approach to these estimations is three steps of calibrations that obtain different characteristics of the measurement system. Details of these calibrations are discussed further below.

In practical telemedicine, the users need to do calibrations and airway measurements themselves, by assembling the system components (i.e., device 10, adaptor 50, conduit 60, and mouthpiece 70) in different ways. However, these assemblies may cause slightly different placements of system components: the adaptor may be sleeved to different positions, and the conduit may be slightly tilted or bent. Such different placements could lead to mismatching between calibration and use setups, introducing measurement errors. In particular, since the WA algorithm iteratively calculates the CSA of airway segments, measurement error will accumulate in calculations and be amplified in lower airway segments. Addressing this problem requires measuring the tiny difference between different system placements, which is difficult. Instead, our solution is to improve the calibration by applying random noise to the original calibration data and constructing a calibration data library. In each measurement, we apply all calibration data in the library to the received signal and select the result with the highest quality. Details of such selection are discussed further below.

Addressing Human Factors—When system 30 is used in telemedicine by different subject groups who differ in physiological conditions and behavior patterns, human factors such as oral movements and breathing sounds may affect airway measurements and thus their potential impacts need to be minimized. In regard to oral movements, the outlet of the mouthpiece 70 should ideally be aligned to the subject's throat and not subject to unintentional oral movements by the subject so that acoustic signals can be smoothly transmitted into the airway. Features of mouthpiece 70 previously discussed (e.g., incisor stopper 76, tongue depressor 78) are provided to prevent unwanted movements of mouthpiece 70 during sampling. In regard to breathing sounds, when users breathe through the interface 40 the breathing airflow goes through the microphone 16 of the electronic device 10 and may hence produce audible sounds that affect airway measurement accuracy. To minimize impact, the received signal strength between the transmitted signal pulses at runtime can be measured, to detect such breathing sounds and issue a warning to the user via the software application for slower breaths. Any remaining breathing sound(s) may be removed by a digital Wiener filter.

Pulmonary Disease Evaluation—With airway CSA measurements, embodiments of the present invention utilize a multi-task learning model to enable both disease prediction and lung function estimation. A major challenge is high variability of airway measurements, even on the same subject in one use of system 30. Such variability is caused by both system noise and physiological airway movements during measurements. It weakens the correlation between airway measurements and disease symptoms, and hence makes it difficult for ML models to make predictions from any single airway measurement. In an example embodiment of the present invention, such problem is addressed by first constructing high-dimensional input data from multiple CSA measurements, to eliminate the aforementioned variability. Then, self-supervised learning is used first to reduce the learning difficulty by extracting distinctive features, and then domain knowledge provided by the user's spirometry results is incorporated into the neural network (NN) model training. Details of such multi-task learning are discussed further below.

System Calibrations—In this section, technical details about the system calibrations that ensure accurate airway measurements by eliminating the impact of various system factors are provided. The impact of source reflection is removed using three steps of calibrations that estimate X(s), Hₚ(s) and Hₛ(s). In the first step, the interface conduit 60 is replaced with a sufficiently long tube (e.g., >5 m). The microphone 16 of the electronic device 10 will then receive no reflection signal but only the transmitted signal from speaker 14, ensuring precise estimations of X(s). Note that since X(s) only relates to characteristics of the smartphone speaker and microphone (or other similar device), this step only needs to be done once on each device. In the second and third steps, the normal interface conduit 60 is used but without the mouthpiece 70, with the second step carried out with the end of the conduit 60 blocked by a hand, and with the third step carried out with the hand removed from conduit 60. Such second and third steps give a fully positive reflection (i.e., Hₒ(s)=1) and a fully negative reflection (i.e., Hₒ(s)=−1) of incident signal, respectively. Denoting the received signal in these two steps as Y₁(s) and Y₂(s), we have:

$${Y}_{1} ( s ) = X ( s ) \frac{1 + {{H}_{p} ( s )}^{2}}{1 - {H}_{p}^{2} ( s ) {H}_{s} ( s )} ,$$

$${Y}_{2} ( s ) = \frac{1 - {{H}_{p} ( s )}^{2}}{1 + {H}_{p}^{2} ( s ) {H}_{s} ( s )}$$

### Therefore, we can get:

$${{H}_{p} ( s )}^{2} = \frac{[ {Y}_{1} ( s ) + {Y}_{2} ( s ) ] X ( s ) - 2 {Y}_{1} ( s ) {Y}_{2} ( s )}{[ {Y}_{1} ( s ) - {Y}_{2} ( s ) ] X ( s )} ,$$

$${H}_{p}^{2} ( s ) {H}_{s} ( s ) = \frac{{Y}_{1} ( s ) + {Y}_{2} ( s ) - 2 X ( s )}{{Y}_{1} ( s ) - {Y}_{2} ( s )}$$

from where we can compute Hₚ(s) and Hₛ(s) from X(s), Y₁(s) and Y₂(s).

Selecting the Best Calibration Data—When we apply all data in the calibration data library to the received signal, we obtain different airway measurements and select the one with the highest quality. We evaluate the quality of a measurement by comparing it with the reference airway CSA curve used in clinical ART. As shown in FIG. 13, the reference curve clearly indicates airway physiological structures, including the oral cavity, oropharyngeal junction (Oroph. J.), pharynx, glottis, and trachea. However, simple distance-based similarity metrics cannot be adopted, because of the heterogeneity of airway lengths in different user groups. Instead, we use dynamic time warping (DTW) to stretch different airway measurements and align them to the same scale. DTW calculates the best match between two given sequences with the minimum mean squared error (MSE), and a similarity score between 0 and 100 can be calculated from the MSE to indicate the measurement quality.

Solely using such similarity scores to evaluate measurement quality may not be always reliable in practice. Due to the DTW's stretching mechanism, some measurements may have high quality scores but still contain large errors. To address this limitation and ensure reliability, we further use a neural network (NN) classifier to identify unacceptable airway measurements. The training data is a small amount of CSA measurements from different individuals and we manually label these data's quality as acceptable or unacceptable. Then, we only accept an airway measurement for disease evaluation if it has high quality score and passes the NN classifier's test.

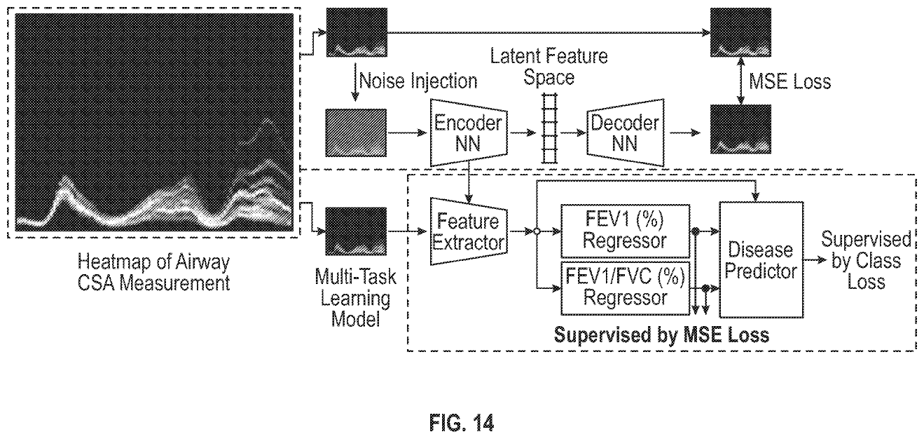

Multi-Task Learning for Pulmonary Disease Evaluation—As shown in FIG. 14, to reduce the learning difficulty caused by high variability of airway measurements, multiple airway CSA measurements of a subject are converted into a heatmap as high-dimensional input data. The multi-task learning model then consists of a feature extractor, two regressors that estimate lung function indices, and a predictor that gives disease predictions.

Constructing High-Dimensional Input—To construct the heatmap as high-dimensional input data, the multiple CSA measurements are considered at each airway position as a distribution of discrete samples, and non-parameterized estimation is conducted to convert these samples into a continuous function that depicts airway dimensions. The heatmap is then produced by concatenating such estimated functions across the entire airway. The heatmaps are then used as one-channel images for the ML model input.

Training the Feature Extractor—A symmetric encoder-decoder architecture is used to train a convolutional auto-encoder as a prerequisite step, and then the trained encoder is used as the feature extractor in later training of the multi-task learning model. The key challenge of training the feature extractor is overfitting due to the limited amount of available input data from human subjects. To address this challenge, self-supervised learning is leveraged to blur the input heatmaps with random Gaussian noise, and learning objective is set as restoring the original input heatmap, by using the mean-square error (MSE) between the restored and original heatmaps as the loss function. In this way, the encoder automatically learns to the representative features that are sufficiently informative for the decoder to restore the original heatmap.

Training the Lung Function Estimators & Disease Predictor—To ensure informative training feedback, the basic rationale is that the present invention's airway measurement and traditional spirometry provide two different modalities for measuring pulmonary disease conditions and complement each other. Hence, the user's spirometry results in his/her health records can be used as pre-known domain knowledge to supervise the training of the ML model. More specifically, the extracted features are used as the input to two regressors that predict the user's FEV1 and FEV1/FVC percentile, the two most representative lung function indices, respectively. The regressors' outputs are supervised by the MSE loss from the user's spirometry results, and are also used as the input to the disease predictor that estimates the corresponding pulmonary disease probability of the user. Note that, the user's spirometry results in health records are only used as the domain knowledge to supervise training. After training completes, the trained regressors and predictor will only take the features from the feature extractor as input, when they are being used in inference for disease evaluation.

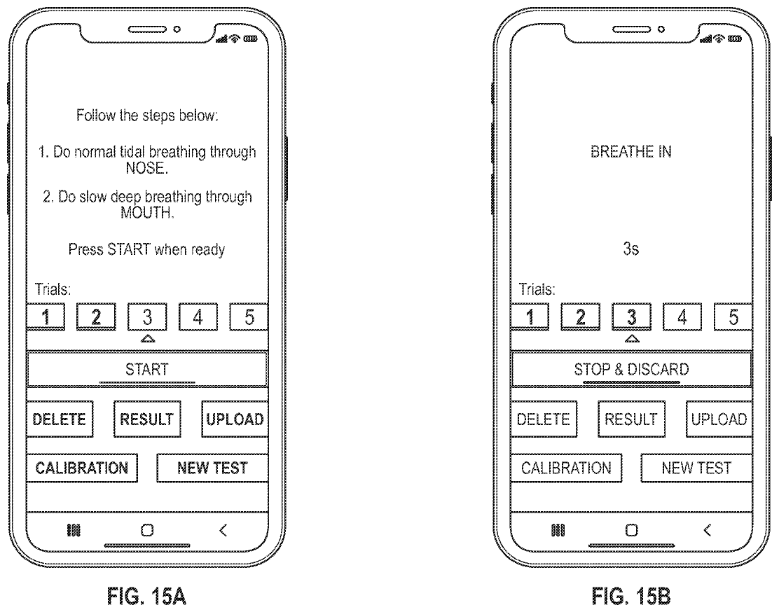

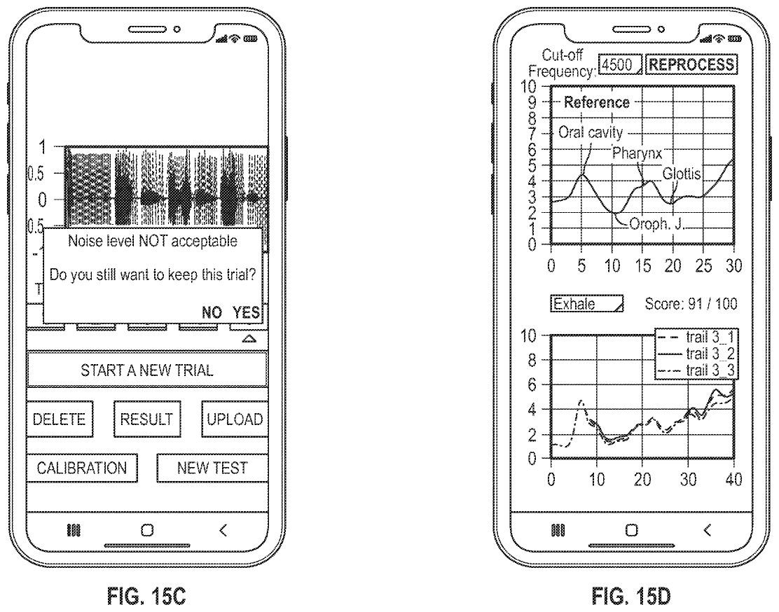

Implementation—In an example embodiment of the present invention such as shown in FIGS. 15A-15D, the methodology discussed herein was implemented as a smartphone app, which senses the airway, analyzes the received signal, and uploads data to a remote server. Before each airway measurement, text instructions are displayed on the screen (not numbered) of the smartphone. During the measurement(s), instructions provided to the user instruct the user to inhale or exhale multiple times with a countdown timer indicating how long particular actions are to be carried out. After each measurement, the app shows the measurement results and can warn the user if loud breathing sounds were produced or if other potential problems/sources of error are detected. After a user completes the whole test, the airway CSA measurements are provided to the ML model which can be deployed locally (e.g., on the smartphone) or remotely (e.g., on a cloud server). In an example embodiment, each module of the ML model contains 3 fully-connected or convolutional layers, depending on the input. In an example embodiment, the ML model was trained using Adam optimizer with a step size of 0.001 and batch size of 32.

From the foregoing it is thus to be appreciated that embodiments of the present invention provide important telemedicine tools and methods to assist clinical decisions in pulmonary disease management. Such arrangements and methods can be generally effortlessly and conveniently used out of clinic whenever needed at relatively minimal cost. Being different from traditional PFT methods such as spirometry, embodiments of the present invention do not require any forced maneuvers or difficult protocols, and can be used during normal breaths. Such arrangements and methods are adaptive as potential impacts from various system and human factors are removed, making embodiments readily applicable to different smartphone models and environmental settings.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method of determining the presence and/or severity of a pulmonary disease in an individual, the method comprising:
directing an ultrasound signal generated by a speaker (14) of an electronic device (10) into an airway of the individual through an interface (40) coupled to the device, wherein the interface has a mouthpiece (70) structured to be received in a mouth of the individual;
receiving with a microphone (16) of the electronic device a reflected version of the ultrasound signal from the airway; and
determining from the reflected version of the ultrasound signal the presence and/or severity of the pulmonary disease in the individual,
wherein the presence and/or severity of the pulmonary disease is determined using a trained machine learning model of a controller (12) of the electronic device.

2. The method of claim 1, wherein directing the ultrasound signal generated by the speaker of the electronic device into the airway of the individual comprises directing a series of acoustic pulses into the airway, and wherein receiving with the microphone of the device the reflected version of the ultrasound signal from the airway comprises receiving a reflected version of the series of acoustic pulses from the airway.

3. The method of claim 1, further comprising filtering and denoising the reflected version of the ultrasound signal with the controller to produce an adjusted reflected signal, and wherein the presence and/or severity of the pulmonary disease in the individual is then determined by the controller from the adjusted reflected signal.

4. The method of claim 1, wherein the electronic device comprises a smartphone.

5. The method of claim 1, wherein the mouthpiece comprises:
a body (72);
a passage (74) defined within, and extending through the body from an inlet (74A) that is coupled to the second end of the interface tube to an outlet (74B) opposite the inlet; and
an incisor stopper (76) disposed adjacent the inlet, the incisor stopper being structured to engage incisors of the individual to fix the orientation of the body and thus the mouthpiece when the individual bites down on the body.

6. The method of claim 5, wherein the mouthpiece further comprises a tongue depressor (78) positioned at the bottom of the body adjacent the outlet that is positioned and structured to be engaged by a tongue of the individual.

7. The method of claim 6, wherein the body, the incisor stopper, and the tongue depressor are each portions of a unitary member.

8. A system (30) for determining the presence and/or severity of a pulmonary disease in an individual, the system comprising:
an electronic device (10) comprising:
a housing (17);
a speaker (14) positioned on or in the housing;
a microphone (16) positioned on or in the housing; and
a controller (12) in communication with the speaker and the microphone; and
an interface (40) structured to convey ultrasound signals produced by the speaker to the airway of the individual and from the airway of the individual to the microphone,
wherein the controller is structured and configured for:
transmitting an ultrasound signal from the speaker to an airway of the individual via the interface;
receiving from the microphone a reflected version of the ultrasound signal from the interface reflected from the airway of the individual; and
determining from the reflected signal the presence and/or severity of the pulmonary disease in the individual, and
wherein the controller is structured and configured for determining the presence and/or severity of the pulmonary disease from the reflected signal using a trained machine learning model.

9. The system of claim 8, wherein the controller is further structured and configured for filtering and denoising the reflected version of the ultrasound signal to produce an adjusted reflected signal, and for determining the presence and/or severity of the pulmonary disease in the individual from the adjusted reflected signal.

10. The system of claim 8, wherein the electronic device is a smartphone.

11. The system of claim 8, wherein the interface comprises:
an adaptor (50) having a body (52) generally defining an interior void (54) therein, the body having a first portion (52A) and a second portion (52B), the first portion being engaged on and around an end of the electronic device such that openings in the housing for the speaker and the microphone are encompassed by body and open into the interior void;
a mouthpiece (70) structured to be positioned in the mouth of the individual; and
an interface tube (60) having a first end (60A) coupled to the second portion of the adaptor and an opposite second end (60B) coupled to the mouthpiece.

12. The system of claim 11, wherein the mouthpiece comprises:
a body (72);
a passage (74) defined within, and extending through the body from an inlet (74A) that is coupled to the second end of the interface tube to an outlet (74B) opposite the inlet; and
an incisor stopper (76) disposed adjacent the inlet, the incisor stopper being structured to engage incisors of the individual to fix the orientation of the body and thus the mouthpiece when the individual bites down on the body.

13. The system of claim 12, wherein the mouthpiece further comprises a tongue depressor (78) positioned at the bottom of the body adjacent the outlet that is positioned and structured to be engaged by a tongue of the individual.

14. The system of claim 13, wherein the body comprises a unitary member and wherein the body, the incisor stopper, and the tongue depressor are each portions of the unitary member.

15. An interface (40) for use with an electronic device (10) for determining the presence and/or severity of a pulmonary disease in an individual, the interface comprising:
a mouthpiece (70) structured to be positioned in the mouth of the individual, the mouthpiece comprising:
a body (72);
a passage (74) defined within, and extending through the body from an inlet (74A) to an outlet (74B) opposite the inlet; and
an incisor stopper (76) disposed adjacent the inlet, the incisor stopper being structured to fix the orientation of the body and thus the mouthpiece when the individual bites down on the body.

16. The interface of claim 15, wherein the mouthpiece further comprises a tongue depressor (78) positioned at the bottom of the body adjacent the outlet and that is structured to be engaged by the tongue of the individual.

17. The interface of claim 16, wherein the body comprises a unitary member and wherein the body, the incisor stopper, and the tongue depressor are each portions of the unitary member.

18. The interface of claim 15, further comprising:
an adaptor (50) having a body (52) generally defining an interior void (54) therein, the body having a first portion (52A) and a second portion (52B), the first portion structured to be engaged on and around an end of the electronic device such that openings in the housing for the speaker and the microphone are encompassed by the body and open into the interior void; and
an interface tube (60) having a first end (60A) coupled to the second portion of the adaptor and an opposite second end (60B) coupled to the inlet of the mouthpiece.
